(19) United States
(12) Patent Application Publication
(10) Pub. No.: US 2024/0420559 A1
(43) Pub. Date: Dec. 19, 2024

(54) SYSTEMS AND METHODS FOR DETECTING HANDWASHING

(71) Applicant: TSUS SASU, Neuilly (FR)

(72) Inventor: Thomas Serval, Neuilly (FR)

(21) Appl. No.: 18/090,020

(22) PCT Filed: Jul. 6, 2021
(86) PCT No.: PCT/IB2021/056061

§ 371 Date: Dec. 28, 2022

Related U.S. Application Data
(60) Provisional application No. 63/048,453, filed on Jul. 6, 2020.

(51) Int. Cl. G08B 21/24; A61B 5/00

(52) U.S. Cl. CPC G08B 21/245; A61B 5/681

(57) ABSTRACT

A method includes (i) receiving data associated with an individual, the individual having hands; (ii) determining, from the received data, that the individual is washing the hands; and (iii) generating a response to be communicated to the individual based at least in part on the determination that the individual is washing the hands.

### CROSS-REFERENCE TO RELATED APPLICATIONS

This application claims the benefit of and priority to U.S. Provisional Application No. 63/048,453, filed Jul. 6, 2020, which is hereby incorporated by reference herein in its entirety.

### TECHNICAL FIELD

The present disclosure relates generally to systems and methods for preventative treatments and more specifically to monitoring and reminding individuals to wash their hands based on healthcare guidelines.

### BACKGROUND

Preventative healthcare includes measures taken to prevent disease. Diseased conditions can be effected by different factors which may include environmental factors, genetic factors, lifestyle choices, disease agents (e.g., bacteria, viruses, algae, etc.), etc. Preventative healthcare includes anticipatory actions that can be taken by individuals to reduce contributory effects of the different factors to a diseased condition. Disease prevention can involve clinical and non-clinical methods. Non-clinical disease prevention can include lifestyle choices such as eating nutritious meals, daily exercises, and improved personal hygiene. Since these lifestyle choices can sometimes require diligence over extended periods of time, and humans, in general, are known to waver from time to time, it would be advantageous to develop tools that buttress individual diligence. The present disclosure is directed to solving problems related to diligence on personal hygiene and other problems.

### SUMMARY

According to some implementations of the present disclosure, a method includes (i) receiving data associated with an individual, the individual having hands; (ii) determining, from the received data, that the individual is washing the hands; and (iii) generating a response to be communicated to the individual based at least in part on the determination that the individual is washing the hands.

According to some implementations of the present disclosure, a method includes (i) receiving data associated with an individual, the individual having hands; (ii) determining, from the received data, that the individual should wash the hands; and (iii) generating a response to be communicated to the individual based at least in part on the determination that the individual should wash the hands.

According to some implementations of the present disclosure, a system for monitoring handwashing includes a sensor, a memory storing machine-readable instructions, and a control system. The sensor is configured to generate data associated with an individual. The individual has hands. The control system includes one or more processors configured to execute the machine-readable instructions to: (a) determine, from the received data, that the individual is washing the hands; and (b) generate a response to be communicated to the individual based at least in part on the determination that the individual is washing the hands.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure.

FIG. 2 is an example of a bathroom according to some implementations of the disclosure.

FIG. 3 is an example flow diagram for detecting handwashing according to some implementations of the present disclosure.

FIG. 4 is an example screenshot for image analysis according to some implementations of the disclosure.

FIGS. 5A and 5B are example screen displays to provide to individuals prior to and during handwashing.

FIG. 6 is an example flow diagram for determining whether an individual should wash her hands, according to some implementations of the present disclosure.

FIG. 7A is an example dashboard for tracking handwashing activities, according to some implementations of the present disclosure.

FIG. 7B is another example dashboard for tracking handwashing activities, according to some implementations of the present disclosure.

FIG. 8A is a screenshot of a first step for a handwashing event, according to some implementations of the present disclosure.

FIG. 8B is a screenshot of a second step for the handwashing event of FIG. 8A.

FIG. 8C is a screenshot of a third step for the handwashing event of FIG. 8A.

FIG. 8D is a screenshot of a fourth step for the handwashing event of FIG. 8A.

FIG. 8E is a screenshot of a fifth step for the handwashing event of FIG. 8A.

FIG. 8F is a screenshot of a sixth step for the handwashing event of FIG. 8A.

FIG. 8G is a screenshot of a seventh step for the handwashing event of FIG. 8A.

FIG. 8H is a screenshot of an eighth step for the handwashing event of FIG. 8A.

FIG. 8I is a screenshot of a ninth step for the handwashing event of FIG. 8A.

FIG. 8J is a screenshot of a tenth step for the handwashing event of FIG. 8A.

FIG. 8K is a screenshot of an eleventh step for the handwashing event of FIG. 8A.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

According to the United States Centers for Disease Control and Prevention (CDC), handwashing is one of the best ways to protect oneself and one's immediate associates from getting sick. Handwashing is known to reduce and/or prevent spread of respiratory and/or diarrheal infections from one person to the next. Handwashing targets disease agents, generally referred herein as germs, which can spread from person to person or from surface to person. Germs can be transferred from a surface to a person when the person touches the surface and then touches her eyes, nose, and/or mouth with unwashed hands. Unwashed hands can further disseminate germs when a person with unwashed hands prepares food or drinks for herself or others.

With humans spread across the globe, disease can easily be spread from one side of the globe to another. Human mobility due to involvement in certain industries can facilitate spread of disease due to unhealthy practices surrounding handwashing. The hospitality industry can cater to many different customers and can become a place where ideas and germs can spread. Examples of the hospitality industry include lodging businesses like hotels, motels, hostels, campgrounds, etc.; transportation businesses like cabs, trains, planes, buses, etc.; entertainment businesses like movie theatres, zoos, museums, spectator sports, etc.; and food businesses like restaurants, food trucks, food stands, grocery stores, etc. To reduce the spread of germs, proper technique and emphasis on handwashing is suggested. The hospitality industry is merely used as an example, but other industries like education and healthcare industry can benefit from proper handwashing techniques as well. With social interactions in school, hospitals, cafeterias, etc., proper handwashing techniques can reduce transmission of diseases.

Washing one's hands properly is the best way to protect oneself from disease caused by germs. The CDC provides a guideline for when handwashing should be performed. These include: (a) before, during, and after preparing food; (b) before eating food; (c) before and after caring for someone at home who is sick with vomiting or diarrhea; (d) before and after treating a cut or wound; (e) after using the toilet; (f) after changing diapers or cleaning up a child who has used the toilet; (g) after blowing one's nose, coughing, or sneezing; (h) after touching an animal, animal feed, or animal waste; (i) after handling pet food or pet treats; and (j) after touching garbage. The World Health Organization (WHO) also has handwashing guidelines showing a proper way to wash hands in order to prevent disease transmission and diseased conditions. Handwashing guidelines and practices are different for the general public when compared to healthcare workers. Furthermore, guidelines can change over time as the environmental landscape changes. For example, types of soaps recommended for use during handwashing can be adjusted over time, recommended use of antiseptics and/or alcohol can change over time, hand drying techniques, and even handwashing techniques can also change over time. As such, some implementations of the disclosure provide an advantage of keeping track of localized handwashing rules and/or globalized recommended handwashing rules to help prevent diseases in different contexts, be it at home or at a hospital.

Some implementations of the present disclosure provide systems and methods for encouraging individual diligence in handwashing by using computer technology. Individual handwashing can be monitored and tracked thus providing a record that can be used to generate appropriate schedules of handwashing. Monitored data can be stored and prepared in a manner such that tracking handwashing can be performed in real-time, whereby immediate feedback can be provided to an individual. Handwashing is a preventative measure or a preventative treatment, and as such, some implementations of the present disclosure alert of “treatment” schedules for the individual based on the individual's profile. For example, a healthcare worker seeing a patient has stricter handwashing requirements than the patient. The patient's handwashing profile and recommendations can be different from the healthcare worker's.

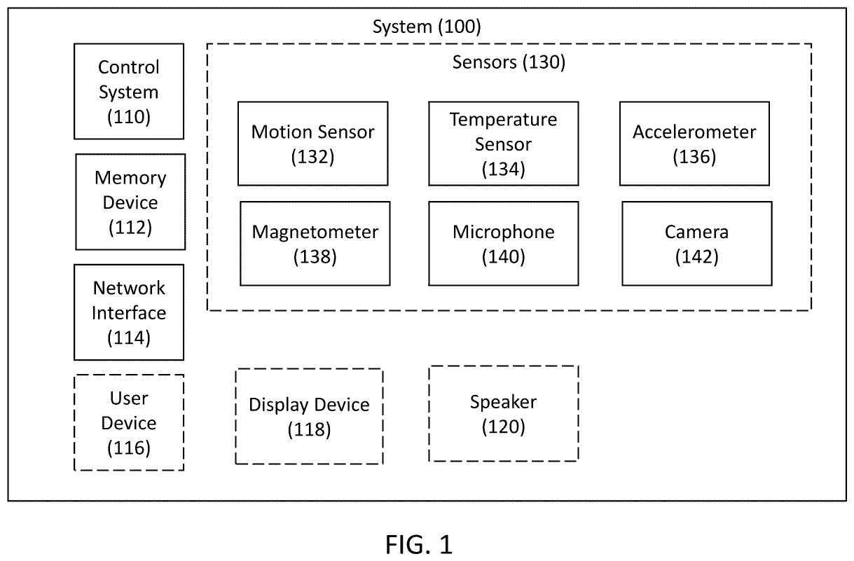

Referring to FIG. 1, a system 100, according to some implementations of the present disclosure, is illustrated. The system 100 can include a control system 110, a memory device 112, a network interface 114, a user device 116, a display device 118, a speaker 120, and one or more sensors 130.

The control system 110 includes one or more processors. The control system 110 is generally used to control (e.g., actuate) the various components of the system 100 and/or analyze data obtained and/or generated by the components of the system 100. The one or more processors of the control system 110 can be a general or special purpose processor or microprocessor. The control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 116, and/or a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other. One or more of the sensors 130 can be coupled to the user device 116. For example, one or more of the sensors 130 can be included in a smartwatch, a smart phone, a smart speaker, a game console, a smart television, a display, jewelry, etc.

The memory device 112 stores machine-readable instructions that are executable by the one or more processors of the control system 110. The memory device 112 can be any suitable computer readable storage device or media, such as, for example, a random or serial access memory device, a hard drive, a solid state drive, a flash memory device, etc. While one memory device 112 is shown in FIG. 1, the system 100 can include any suitable number of memory devices 112 (e.g., one memory device, two memory devices, five memory devices, ten memory devices, etc.). The memory device 112 can be coupled to and/or positioned within a housing of the user device 116, within a housing of one or more of the sensors 130, or any combination thereof. Like the control system 110, the memory device 112 can be centralized (within one such housing) or decentralized (within two or more of such housings, which are physically distinct).

The network interface 114 is configured to receive data from the one or more sensors 130 such that the data can be stored in the memory device 112 and/or analyzed by the one or more processors of the control system 110. The network interface 114 can communicate with the one or more sensors 130 using a wired connection or a wireless connection (e.g., using an RF communication protocol, a WiFi communication protocol, a Bluetooth communication protocol, over a cellular network, etc.). The network interface 114 can include a serial bus interface, a parallel bus interface, an antenna, a receiver (e.g., an RF receiver), a transmitter (e.g., an RF transmitter), a transceiver, or any combination thereof. The network interface 114 can also include one more processors and/or one more memory devices that are the same as, or similar to, the one or more processors of the control system 110 and the memory device 112 described herein. In some implementations, the network interface 114 is coupled to or integrated in the user device 116. In other implementations, the network interface 114 is coupled to or integrated (e.g., in a housing) with the control system 110 and/or the memory device 112.

The display device 118 is generally used to display image(s) including still images, video images, or both and/or information regarding a handwashing process. For example, the display device 118 can provide information regarding the status of duration elapsed since the handwashing began, a duration remaining until the recommended handwashing duration is reached, a notification indicating that an individual wash her hands, etc., In some implementations, the display device 118 acts as a human-machine interface (HMI) that includes a graphic user interface (GUI) configured to display the image(s) as an input interface. The display device 118 can be an LED display, an OLED display, an LCD display, or the like. The input interface can be, for example, a touchscreen or touch-sensitive substrate. In some implementations, the display device 118 can be coupled to or integrated in the user device 116. In some implementations, the display device 118 can be coupled to or integrated (e.g., in a housing) with the control system 110 and/or the memory device 112. In some implementations, the display device 118 can be coupled to or integrated in an electronic device that houses the sensors 130.

The speaker 120 outputs sound waves that are audible to an individual. The speaker 120 can be used, for example, as an alarm clock or to play an alert or message to the individual (e.g., in response to an event). The speaker 120 can be coupled to or integrated in the user device 116 or some other electronic device that houses the sensors 130.

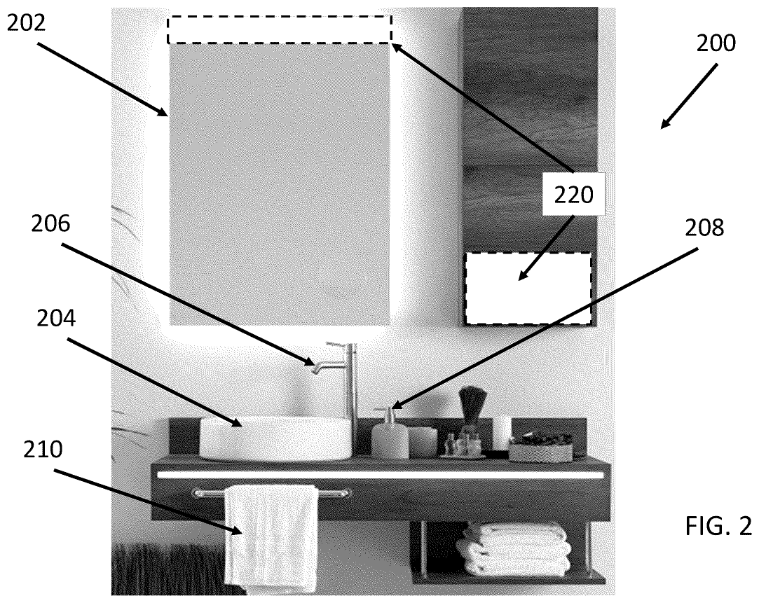

Referring to FIG. 2, in some implementations, the system 100 can be used in a bathroom 200. The bathroom 200 can include a bathroom mirror 202, a bathroom sink 204, a faucet 206, a soap dispenser 208, one or more hand towels 210, and one or more locations 220 where one or more components of the system 100 can be affixed or mounted. In some implementations, the one or more components of the system 100 can be affixed or mounted to the faucet 206. In a simple procedure, an individual washing her hands turns on the faucet 206, uses the soap dispenser 208 to place soap on her hands, rubs her hands together for at least a prescribed duration, rinses her hands under the faucet 206, and turns off the faucet 206. She then dries off using the towel 210. In some implementations, this simple procedure is captured by the system 100 using the user device 116 in the form of a smart watch on the individual's wrist. In some implementations, the simple procedure is captured by the system 100 using a camera, a smart phone, a portable game system, or some other electronic device mounted on at least one of the one or more locations 220. In some implementations, the simple procedure is captured by the system 100 using a smart phone in a pocket of the individual.

In some implementations, components of the system are mounted in hospitals or other places that provide a visual indicator (e.g., a red light is lit) when the individual starts washing her hands and another visual indicator (e.g., a green light is lit) when the individual finishes washing her hands. In some implementations, once the individual is finished, a chime or some other auditory indicator is provided.

In some implementations, one or more cameras are mounted at one or more of the locations 220. The one or more cameras can be mounted above the faucet 206 and/or above the mirror 202, pointing downward toward the sink 204. In some implementations, a smart phone camera is used such that the smart phone is mounted to the mirror 202 using a mount attached to the smart phone. An example mount can be a suction cup on one side configured to engage with a surface of the mirror. On the other side, a rod or two can be configured to hold the smart phone in place. In some implementations, more than one camera or smart phone is mounted in the bathroom 200 so that a three dimensional model of the hands is obtained. In some implementations, a mounted smart phone's screen is active so that the individual can view a progress on the screen or can visually engage with the screen or orally engage with the smart phone while handwashing. In some implementations, the visual engagement or oral engagement is based on a handwashing game where points are tallied, thus providing a rewarding experience that can encourage the individual to wash her hands more often.

In some implementations, the one or more cameras are mounted in jewelry or on other items (e.g., a smartwatch) on a person's body. For example, the one or more cameras can be included in one or more bracelets. The bracelets can perform some data processing or can offload some of the processing.

Referring back to FIG. 1, one or more of the sensors 130 include a motion sensor 132, a temperature sensor 134, an accelerometer 136, a magnetometer 138, a microphone 140, a camera 142, or any combination thereof. Generally, each of the sensors 130 is configured to output sensor data that is received and stored in the memory device 112 or one or more other memory devices. While the sensors 130 are shown and described as including each of the motion sensor 132, the temperature sensor 134, the accelerometer 136, the magnetometer 138, the microphone 140, the camera 142, more generally, the sensors 130 can include any combination and any number of each of the sensors described and/or shown herein. The sensors 130 generate data that the control system 110 processes in order to identify and/or characterize an individual's handwashing activities.

The motion sensor 132 outputs motion data that can be stored in the memory device 112 and/or analyzed by the one or more processors of the control system 110. In some implementations, the motion sensor 132 generates motion data indicating that the user device 116 is in shaking in a pocket of an individual. This motion data indicating that the user device 116 is shaking can be combined with sound data present during the shaking. The combined motion and sound data can be analyzed by the control system 110 to determine whether the individual is washing her hands. In some implementations, the motion sensor 132 generates motion data that indicates that the individual is at a bathroom sink, a kitchen sink, or a sink at a healthcare facility. The motion data can be combined with visual data and/or sound data to determine when the individual begins handwashing, when the individual stops handwashing, how long the individual washed her hands, etc. The motion sensor 132 can be realized as an acoustic sensor where a microphone and a speaker are used to sense distance between the individual and the sink. Sound emitted from the speaker is reflected by the environment and picked up by the microphone such that changes in the reflections picked up by the microphone can be attributed to the individual's movement. The motion sensor 132 can be realized as a radio frequency (RF) sensor where an RF transmitter and an RF receiver are used to sense distance between the individual and the sink. Similar to the acoustic sensor, reflected RF waves received at the RF receiver can be monitored such that disturbance in the reflections can be attributed to the individual's movement.

The temperature sensor 134 outputs temperature data that can be stored in the memory device 112 and/or analyzed by the one or more processors of the control system 110. In some implementations, the temperature sensor 134 generates temperatures data indicative of a core body temperature of the individual, a water temperature flowing through the faucet 206, an ambient temperature of the environment or room, or any combination thereof. The temperature sensor 134 can be, for example, a thermocouple sensor, an infrared sensor, an infrared camera, or any combination thereof.

The accelerometer 136 outputs acceleration data that can be stored in the memory device 112 and/or analyzed by the one or more processors of the control system 110. In some implementations, the accelerometer 136 is included in the user device 116 and senses movement of the user device 116. In some implementations, the user device 116 is a smart watch such that the acceleration data indicates that the individual is currently washing her hands. Acceleration data can be motion data as discussed above with respect to the motion sensor 132. The magnetometer 138 outputs force data that can be stored in the memory device 112 and/or analyzed by the one or more processors of the control system 110. In some implementations, the magnetometer 138 is included in the user device 116 and change in the force data provided by the magnetometer 138 can indicative of the individual currently washing her hands. In some implementations, the magnetometer 138 and the accelerometer 136 are both used to generate motion data for monitoring movement during a handwashing event.

The microphone 140 outputs sound data that can be stored in the memory device 112 and/or analyzed by the one or more processors of the control system 110. The microphone 140 can be used to record sounds while an individual is washing her hands to determine whether the individual meets a recommended handwashing standard, as described in further detail herein. The microphone 140 can be coupled to or integrated in the user device 116.

The camera 142 outputs image data reproducible as one or more images (e.g., still images, video images, thermal images, or a combination thereof) that can be stored in the memory device 112. The image data from the camera 142 can be used by the control system 110 to evaluate the individual's handwashing technique, determine when the individual begins washing her hands, determine when the individual stops washing her hands, or any combination thereof. For example, the image data from the camera 142 can be used to identify a location of the individual, to determine a time when the individual arrives at the bathroom sink 204, and to determine a time when the individual turns on the faucet 206, and to determine when the individual dries her hands with the towel 210.

While the control system 110 and the memory device 112 are described and shown in FIG. 1 as being a separate and distinct component of the system 100, in some implementations, the control system 110 and/or the memory device 112 are integrated in the user device 116. Alternatively, in some implementations, the control system 110 or a portion thereof can be located in a cloud (e.g., integrated in a server, integrated in an Internet of Things (IoT) device, connected to the cloud, be subject to edge cloud processing, etc.), located in one or more servers (e.g., remote servers, local servers, etc.), or any combination thereof.

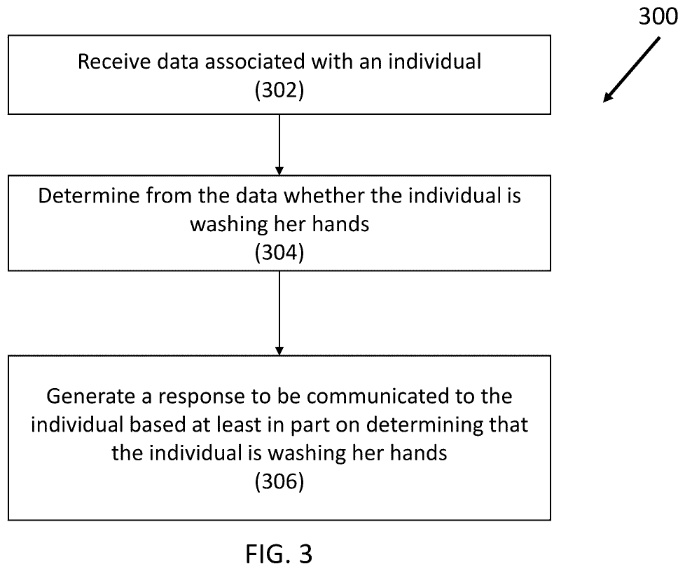

Referring to FIG. 3, a method 400 for tracking and/or monitoring handwashing by an individual is illustrated according to some implementations of the present disclosure. One or more steps of the method 300 can be implemented using any element or aspect of the system 100 (FIG. 1) described herein.

Step 302 of the method 300 includes receiving data associated with an individual. The data received can be data generated by the sensors 130 as described above in connection with FIG. 1. The data received can include motion data, sound data, temperature data, etc. In some implementations, the user device 116 is a wearable device associated with the individual. For example, the user 116 can be a smart watch or a fitness tracker that the individual is wearing. The wearable device can include motion data generated by the motion sensor 132, the accelerometer 136, and/or the magnetometer 138. The wearable device can also include the microphone 140 for generating sound data. The motion data and the sound data can be received by the control system 110.

In some implementations, at step 302, the camera 142 generates image data which is received by the control system 110. In some implementations, the user device 116 provides location data to the control system 110. The location data can be provided using the motion sensor 132 or can be provided using a global positioning system (GPS) receiver embedded in the user device 116. In some implementations, the user device 116 can provide calendar information about the individual. For example, the individual's calendar can indicate that lunch time is nearing for the individual.

At step 304, the control system 110 determines from the received data of step 302 whether the individual is washing her hands. In some implementations, motion data and sound data are combined to determine whether the individual is washing her hands. For example, in the case where the user device 116 is a smart watch, motion data can indicate non-zero acceleration and/or changes in orientation based on magnetic force sensing. When the control system 110 determines that there is nonzero acceleration or that the changes in orientation exceed a threshold, the control system 110 checks the sound data to ensure that the individual is actually handwashing.

The sound data includes sound in the individual's environment. Sound of running water from a faucet can be distinct and can be filtered from the environmental sound data. Sound data from running water can include beginning and ending events where the sound amplitude includes spikes. These spikes may be due to the valves opening and closing in the faucet. The control system 110 can analyze the sound data to identify these amplitude spikes in the sound that correspond to mechanical valves opening and closing, and use these amplitude spikes to estimate start and end times of the individual's handwashing events. The sound data can be of a certain pattern where amplitude of running water starts from an amplitude spike because of opening of the valve for the faucet, continues with a lower amplitude while the water is running, and ends with another amplitude spike because of closing the valve for the faucet.

Sound data obtained by the microphone 140 is linked to a sampling frequency of the sound of running water. For example, the sound of running water can be sampled at 44.1 kHz, thus the control system 110 when performing a Fast Fourier Transform (FFT) of the sound data can sometimes end up with a symmetric data series where the axis of symmetry is around half the sampling frequency (i.e., at 22.05 kHz). An FFT analysis can identify different frequency components in the sound data to verify that the sound data corresponds to running water. In some implementation, the sound data includes multiple frequency modes (e.g., three frequency modes, four frequency modes, five frequency modes, etc.). In an example where five frequency modes are present, example frequencies with FFT-peak amplitudes can include 0.2 kHz, 0.5 kHz, 1.7 kHz, 8 kHz, and 11 kHz. The FFT analysis of sound between the amplitude spikes to identify frequency modes can increase confidence that the amplitude spikes recorded correspond to sound from a faucet and not some other environmental entity.

FFT analysis can also provide insight into flow rate. Once the frequency modes are identified, in some implementations, running water changes pitch as a function of flow rate. For example, in the case where five frequency modes of 0.2 kHz, 0.5 kHz, 1.7 kHz, 8 kHz, and 11 kHz, the amplitude of the 0.2 kHz frequency component can be lower than the amplitude of the 11 kHz frequency component at low flow rates (e.g., about 20 mL/s to 100 mL/s). The amplitude of the 0.2 kHz frequency component can be comparable to the amplitude of the 11 kHz frequency component at medium flow rates (e.g., about 100 mL/s to 150 mL/s). The amplitude of the 0.2 kHz frequency component can be greater than the amplitude of the 11 kHz at high flow rates (e.g., greater than about 150 mL/s). When a faucet is turned on, water flow rate increases until the water flow rate reaches a peak flow rate, and when the faucet is turned off, water flow rate decreases until the flow rate reaches a minimum, preferably 0 mL/s (i.e., a non-leaky faucet). Comparing the FFT amplitudes of the highest and lowest modes, for example, 0.2 kHz and 11 kHz, can provide an indication that the faucet is turned on or that the faucet is turned off.

Sound data combined with motion data can be used to provide other types of information associated with handwashing. An estimate of how long the individual washed her hand can be provided based on a pattern identified in the motion data. For example, if the motion data is motion from a wearable device on the wrist of the individual, sudden, small movements around a period where running water sound is detected can be associated with a handwashing event. Timestamps can be recorded for when the individual started washing her hands based on the motion data, the sound data, or both. Similarly, timestamps can be recorded for when the individual stopped washing her hands based on the motion data, the sound data, or both.

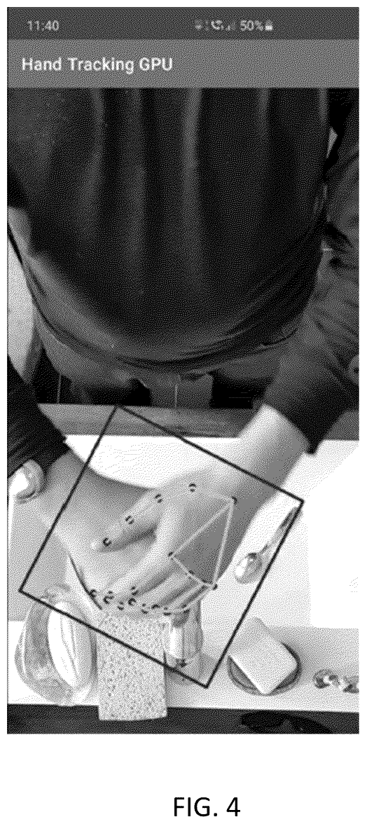

In some implementations, computer vision can be used to ascertain characteristics of handwashing. For example, FIG. 4 illustrates an example where a hand is identified in captured image data. The individual's hands can be identified to determine finger positions during the handwashing process. In some implementations, hand tracking software is used to create a three dimensional model of each hand including the fingers, knuckles, palm, etc. In some implementations, the control system 110 utilizes pattern recognition to determine which captured images includes hands. Hands include at least one digit (or finger), so the pattern recognition algorithm used by the control system 110 can use identified features (e.g., digits, range of motion of the digits, digit curvature, etc.) in the captured images to track the hands. As the individual washes her hands, the control system 110 can determine whether fingers of each hand are interlocking and/or crossing over. Once fingers are identified, the movement of the fingers crisscrossing can be determined more easily than if the fingers were not previously identified. A frame-by-frame comparison of captured images can be used to determine motion of the fingers to determine whether fingers are interlocking. Handwashing usually begins with both hands apart, so the earlier the camera 142 captures the hands, the easier tracking interlocking and crossing over events will be. The control system 110 can also determine how long each interlocking and/or crossing over event lasts. With fingers being identified, skin surfaces of the fingers, palms, back of the hand, etc., can be identified. That way, the control system 110 can determine from the image data which areas on the skin surfaces are crossed over when the individual was handwashing. In some implementations, the control system 110 determines how many times a skin surface is crossed over during handwashing.

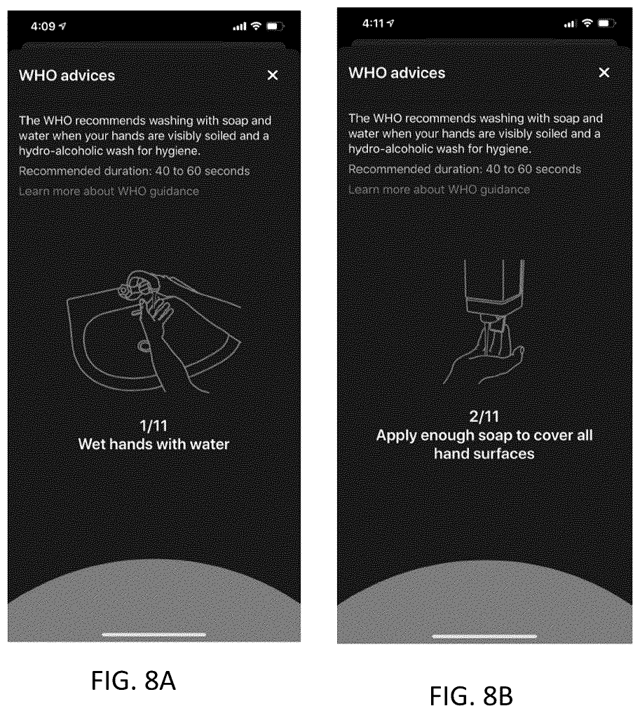

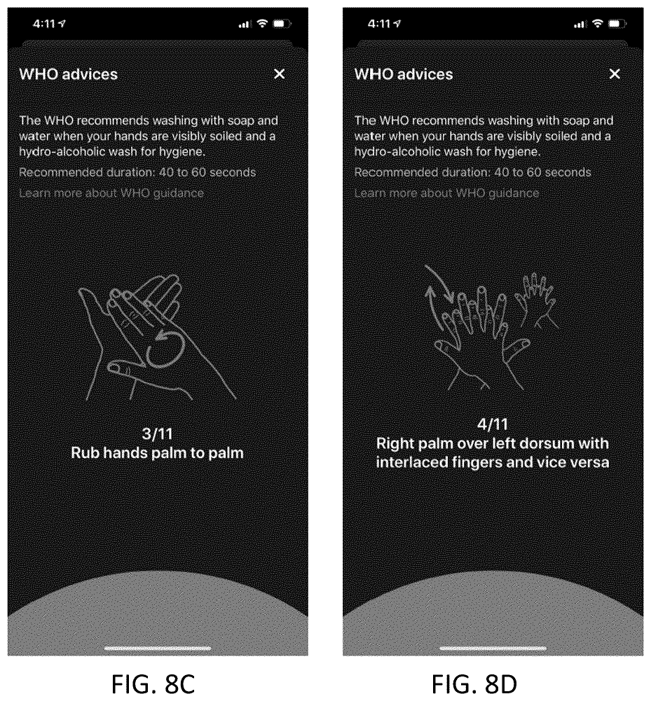

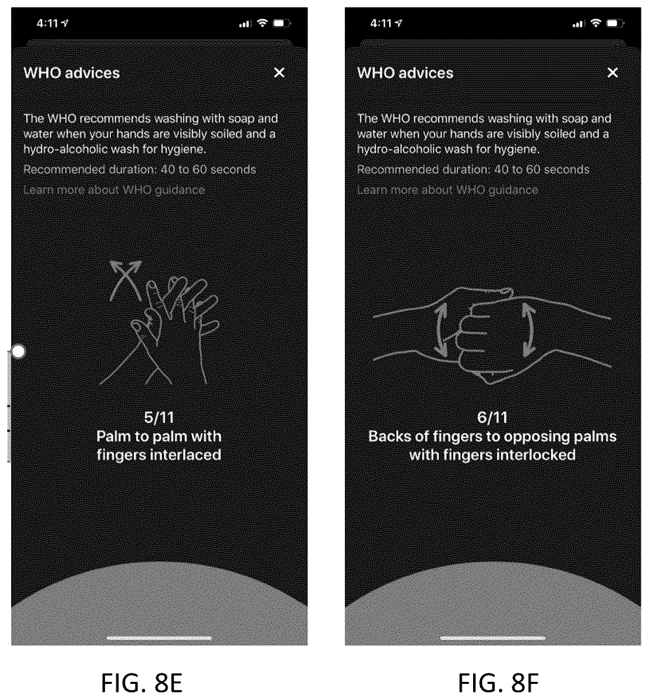

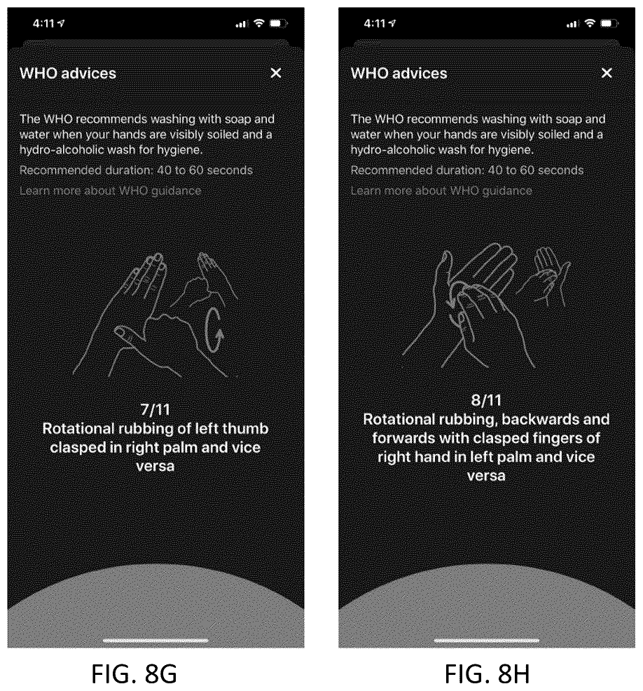

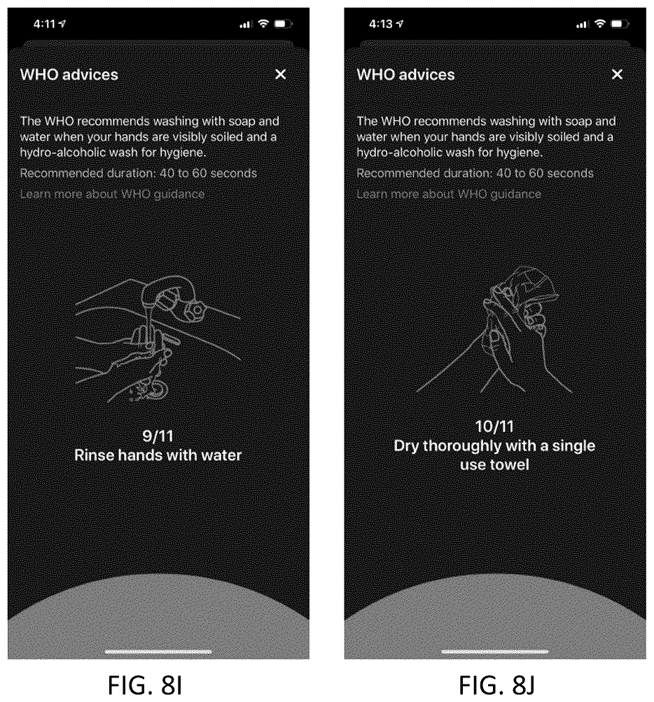

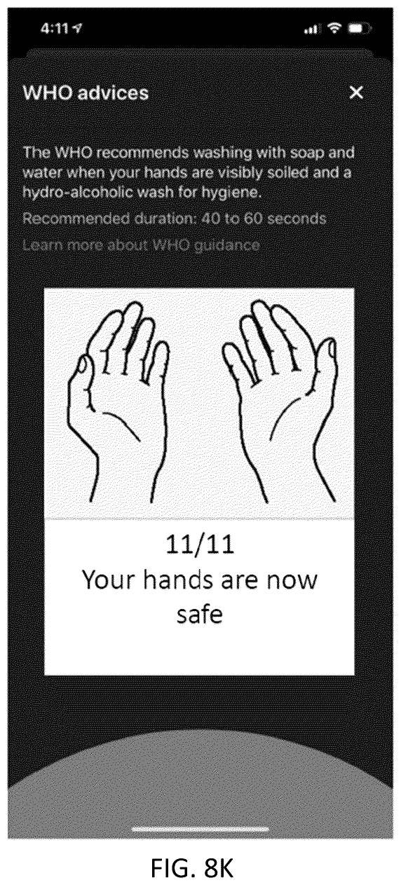

Skin surface crossover can be determined through gross approximations. For example, gesture recognition can be used to approximate skin surface crossed over. A period of time for interlocking fingers can indicate that approximately a percentage of surface between fingers have been crossed over. For example, ten seconds can amount to one hundred percent crossover, one second can amount to forty percent, etc. Palm to palm rubbing gestures can indicate that the skin on the palms have been crossed over. Similarly, a period of time for palm to palm rubbing can be translated to a percentage. Other gestures that can be detected are right palm over left dorsum, left palm over right dorsum, right palm over left dorsum with interlaced fingers, left palm over right dorsum with interlaced fingers, palm to palm with fingers interlaced, backs of fingers to opposing palms with fingers interlocked, etc. In some implementations, a certain set of gestures are a complete set to cover all surfaces on the hands, thus, when the control system 110 detects all gestures in the complete set of gestures, then the control system 110 determines that one hundred percent of skin surface on the hands have been crossed over. In an example, the complete set of gestures can be gestures identified in FIGS. 8A-8H. The complete set of gestures can be updated by healthcare guidelines. Gross approximations can reduce computation complexity in tracking hands while handwashing, facilitating real-time tracking of handwashing.

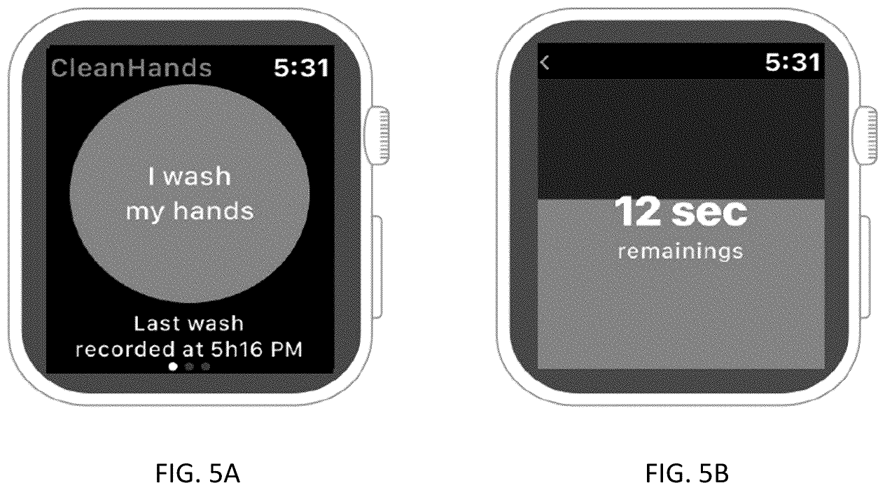

At step 306, a response to be communicated to the individual is generated based at least in part on determining that the individual is washing her hands. In some implementations, when the control system 110 determines that the individual is washing her hands, a status that indicates the last time when the individual washed her hands can be displayed on the user device 116 (see e.g., FIG. 5A). In FIG. 5A, a smart watch with a handwashing app can display “Last wash recorded at 5:16 PM.”

In some implementations, when the control system 110 determines that the individual is washing her hands, a status that indicates how long the handwashing process should take is provided. The recommended time can be based on recommended times by the CDC, the WHO, or any other healthcare oriented organization. For example, in FIG. 5B, a smart watch can display a timer that indicates how many seconds (e.g., twelve seconds) are remaining for the handwashing. The recommended times can be stored in the memory device 112 and updated over time based on updates to healthcare policy changes.

In some implementations, when the control system 110 determines that the individual is washing her hands, a game can pop up on a smart phone and points are awarded depending on how long the individual is washing. The points awarded can be capped once the individual exhausts the recommended duration for handwashing. In some implementations, music is played on the smart phone, the music length coinciding with the recommended duration for handwashing. That way, the individual can use the music as cue for when to stop washing.

In some implementations, when the control system 110 determines that the individual is washing her hands, the speaker 120 provides a chime or a sound when the recommended duration for handwashing is exhausted. In some implementations, the display device 118 flashes to provide a visual indication that the recommended duration for handwashing is exhausted.

In some implementations, the control system 110 generates a report to be sent to the user device 116 associated with the individual. For example, a smart phone or a smart watch of the individual can receive a notification, a text message, a text file, a graphic file, etc. The notification can include a daily summary of handwashing activities performed by the individual. The daily summary can include how long the individual washed their hands for each handwashing event, how many handwashing events during the day, specific times when the individual washed her hands, etc.

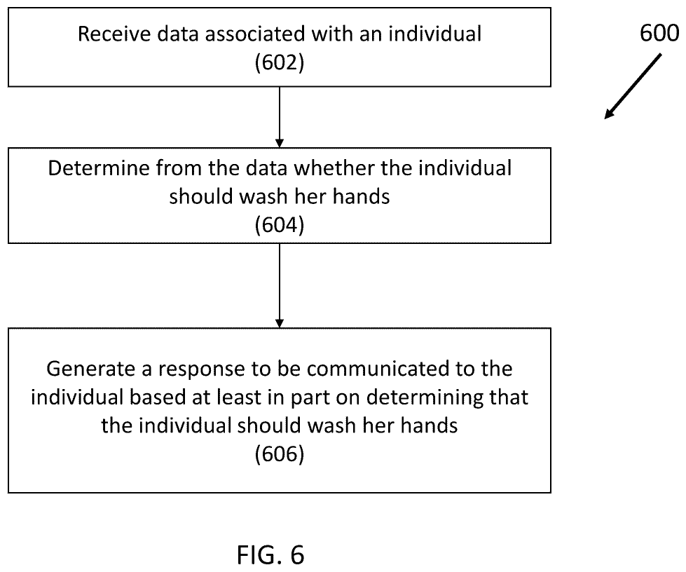

Referring to FIG. 6, a method 600 according to some implementations of the present disclosure is illustrated. The method 600 can be implemented using any combination of elements or aspects of the system 100 described herein.

Step 602 of the method 600 is the same as, or similar to step 302 of the method 300 (FIG. 3) and includes receiving data associated with an individual.

Step 604 involves determining from the received data whether the individual should wash her hands. In some implementations, the received data is a calendar event indicating that the individual will eat soon. For example, the individual is scheduled to have a lunch meeting in ten minutes or that the individual is scheduled to have a dinner meeting in five minutes. As discussed earlier, the CDC recommends handwashing prior to eating, so the control system 110 can determine that the individual should wash her hands based on a time duration before the lunch meeting or dinner meeting being lower than a threshold. For example, being lower than 15 minutes, being lower than 10 minutes, being lower than 5 minutes, etc.

In some implementations, the calendar event indicates that an in-person meeting is over based on an end-time of the meeting and a location indicated in the meeting. Since handshaking is customary in some societies, the control system 110 can determine that the individual should wash her hands. Furthermore, since changes in location to unfamiliar locations or locations that the individual does not frequent can introduce the individual to new kinds of germs that the individual has not encountered, the control system 110 can determine that the individual should wash her hands. For example, the control system 110 can determine after the end-time of the meeting that the individual should wash her hands. The control system 110 can determine that when the individual changes location for a meeting, a reminder for handwashing should be automatically generated in the individual's calendar.

In some implementations, the received data is GPS data from the individual's smart phone or some wearable device. The GPS data can indicate that the individual is taking out the trash based on the individual moving from one location within a living space to an outside location around and back to the living space. Over time, the control system 110 can determine that the individual is taking out the trash and can determine that once the trek inside commences, that the individual should wash her hands. GPS data can also be used in context of unfamiliar location as described above with respect to the individual having a meeting. When the GPS data indicates that the individual is in a new location, the individual has left a work location, a home location, etc., the control system 110 can determine that the individual should wash her hands. In some implementations, geolocation is used based on WiFi connectivity, Bluetooth connectivity, IP address of the individual's smart phone to determine location information like city, ZIP code, or a more specific location (e.g., business name) of the individual.

In some implementations, the received data is sound data from the individual's smart phone or some wearable device. The sound data can indicate that the individual coughed, sneezed, or blew her nose. FFT and time series analyses can be used by the control system 110 to detect these different events. Once the events are detected, the control system 110 can determine that the individual should wash her hands.

In some implementations, the control system 110 determines that the individual should wash her hands after no handwashing activity has been recorded in a certain time duration. In some implementations, the time duration is two hours, three hours, four hours, etc. In some implementations, the control system 110 can have a minimum number of times that the individual should wash her hands, and can determine at certain times during the day that the individual should wash her hands in order to meet the minimum number for the day.

At step 606, the control system 110 generates a response to be communicated to the individual based at least in part on determining that the individual should wash her hands. As discussed in connection with step 306, visual indicators and/or auditory indicators can be provided to the individual using a smart phone or a wearable device. The indicators can be reminders that the individual wash her hands. In some implementations, sound alarms or music are the auditory indicators. In some implementations, a notification is sent to the individual's laptop, smart phone, etc. The notification can include a last time the individual washed her hands, a message that the individual is overdue for a handwashing, a reminder that a handwashing event should be performed, a message indicating a number of handwashing events remaining, etc.

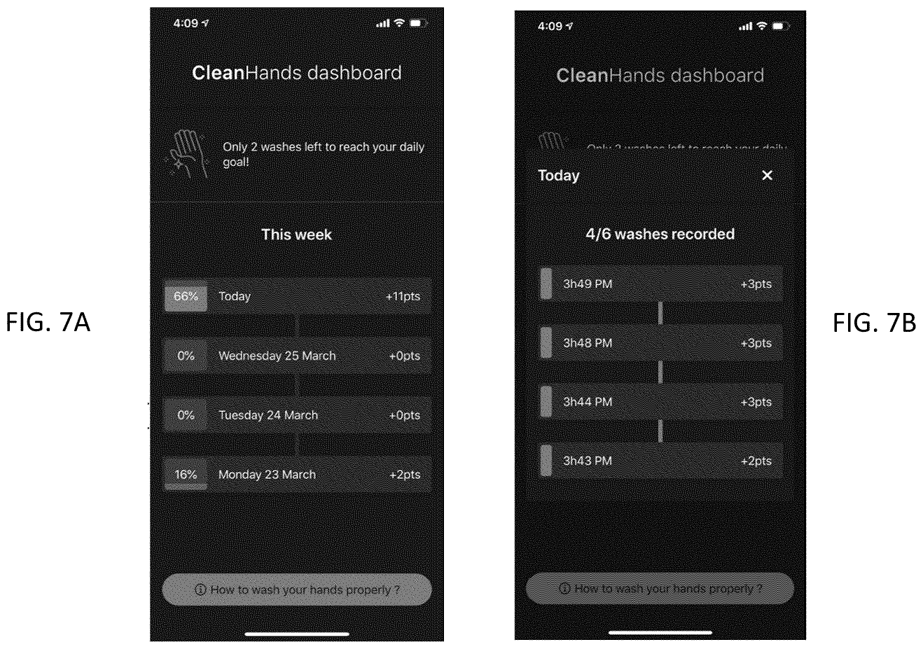

Referring to FIG. 7A, an example dashboard for handwashing activities is illustrated. The dashboard can be shown on a screen of a smart phone. The dashboard can provide a daily handwashing score for each day of the week. For example, +2 pts for Monday 23 March, +0 pts for Tuesday 24 March, etc. The dashboard can also provide a reminder for today indicating that the individual can reach her daily goal. For example, “Only 2 washes left to reach your daily goal!” can be displayed to encourage the individual. The dashboard can also provide a percentage amount on whether the individual met her daily goals. For example, 16% for Monday 23 March, 0% for Tuesday 24 March, etc.

In FIG. 7A, the individual can interact with the dashboard by selecting “Today” which will provide another screen as illustrated in FIG. 7B. Referring to FIG. 7B, specific details concerning handwashing events of the selected day are provided. For example, timestamps of when specific handwashing events can be provided. The number of reward points for each of the handwashing events can be provided as well (e.g., +3 pts for 3:49 PM handwashing event, +2 pts for 3:43 PM handwashing event, etc.). A number of handwashing events can be recorded and compared to a minimum number of handwashing events (e.g., 4/6 washes recorded).

The individual can interact with the dashboard of FIG. 7A further selecting “How to wash your hands properly?” button. In some implementations, an instructional video can be shown to the individual when this button is selected. In some implementations, a slide show can be shown to the individual such that the individual is able to control slide transitions at will. For example, referring to FIGS. 8A-8K, example slides can display WHO handwashing recommendations. Each slide can be numbered with an instruction and with a graphic. For example, FIG. 8A pertains to wetting hands with water, FIG. 8B pertains to applying soap to cover all hand surfaces, FIG. 8C pertains to rubbing hands palm to palm, FIG. 8D pertains to right palm over left dorsum with interlaced fingers and vice versa, FIG. 8E pertains to palm to palm with fingers interlaced, FIG. 8F pertains to backs of fingers to opposing palms with fingers interlocked, FIG. 8G pertains to rotational rubbing of left thumb clasped in right palm and vice versa, FIG. 8H pertains to rotational rubbing backwards and forwards with clasped fingers of right hand in left palm and vice versa, FIG. 8I pertains to rinsing hands with water, and FIG. 8J pertains to drying thoroughly with a single use towel. FIG. 8K is the last step showing that once steps in FIGS. 8A-8J are taken, then the individual's hands will be clean.

Notification settings and other settings for the user device 116 can be set. For example, routine settings can be set for when the individual goes to sleep such that at the individual's bedtime, a reminder is provided to wash her hands. In some implementations, a frequency of reminders can be set such that reminder of WHO recommendations can be provided at every meal or the individual can determine for herself when reminders should be provided throughout the day. During handwashing events, a reminder to use soap or an antiseptic can be provided to the individual. The individual can further select which tutorials to view while handwashing. These may include GIFs, step-by-step videos, musical timers, etc. Before starting a tutorial, a reminder for cleaning and wiping her smart phone, keyboard, mouse, door handle, etc., can be provided to the individual.

Some implementations of the present disclosure provide a watch including an accelerometer and/or a magnetometer in the watch that would monitor movements and/or sound to determine when an individual is washing her hands, and provide feedback to the individual. The accelerometer and/or magnetometer detects movements and a microphone listens for running water and possibly changes in pitch of the running water. Using these measurements, feedback including how long the individual washed for and how often the individual washes her hands can be provided. In some implementations, a location of the individual's smart phone can be used to determine handwashing. For example, the smart phone being in a pocket of the individual can measure vibrations during handwashing such that the vibrations combined with the sound data can be used to determine whether the individual is washing her hands.

Some implementations of the present disclosure use visual modalities like computer vision to determine how well the individual is washing her hands. In an example, hand tracking software is used to generate a three dimensional model of the individual's fingers to see whether the fingers are interlocking and/or crossing over, and for how long. In some implementations, cameras mounted to capture the image for analysis can be from a smart phone so that kids or adults can interact with the smartphone to see the screen of the smart phone, playing a game with the smartphone while washing their hands.

Some implementations of the present disclosure provide monitoring and tracking of handwashing. The individual's handwashing activities are monitored and tracked to determine whether she washed her hands at the appropriate times, how long she washed her hands, etc. The monitored data can be sent by notification (e.g. vibration, sound, light) to the individual's watch or phone when handwashing is completed. Vibration or audio feedback is beneficial if the phone was in a pocket of the individual's clothing or purse. In some implementations, detecting a handwashing activity starts a timer and alerts the individual when the individual is finished washing.

Some implementations of the present disclosure alert individuals when they need to wash their hands using GPS data, calendar information, etc. Personnel can be prevented from entering an area based on whether individuals properly washed their hands. For example, a handwashing station can be set up outside a clinic, and visitors to the clinic may be asked to wash their hands before entering. Some implementations of the present disclosure may be used to track the handwashing techniques of the individuals. If the techniques are not proper, guidance may be provided or the person may be asked to re-wash before being allowed to enter the clinic. Although a clinic is used here as an example, other places like restaurants, food bars, grocery stores, etc. can control who gets to enter based on handwashing technique.

By monitoring handwashing techniques, handwashing habits can be automatically tracked. Some implementations of the present disclosure can replace machines used to test residue on people's hands. For example, in a factory setting, proper handwashing technique can be monitored instead of swiping the hands of employees or other persons on the factory floor to analyze for germs or other residue. Employees that use proper handwashing techniques are determined to have clean hands without having to “sample” the employees' hands. This process can reduce costs associated with adhering with hygiene standards in the factory. Although a factory is used as an example, other places like restaurants, hospitals, food bars, etc. can benefit from such a system.

Along with testing residue, some handwashing methods involve placing paint or fluoride on a person's hands. After handwashing, testing is performed to determine whether there are fluoride or paint residues left on the person's hands. If there is a trace left on the person's hands, then the person's hands are deemed unclean. The use of fluoride is not ideal because it can irritate people's skin. As such, some implementations of the present disclosure can replace these techniques.

In some implementations, a number of gestures can be determined to form a complete set, or a number of gestures can be determined to form minimum threshold for determining that the person's hands are clean. For example, FIGS. 8A-8K provide different gestures that can be sensed by the system 100. In an implementation, all gestures indicated in FIGS. 8A-8K are included in the complete set, hence if all of the eleven gestures are not detected, then the system 100 determines that the person's hands are not clean. In another implementation, six of the gestures are determined to constitute clean hands, therefore, when those six gestures are detected, the person's hands are determined to be clean. For example, FIGS. 8C-8H denote the six gestures being sensed by the system 100, and when all six of those gestures provided in FIGS. 8C-8H are detected, then the person's hands are determined to be clean. In another implementation, a threshold of gestures is determined, and any acceptable gestures are counted against the threshold. For example, if the threshold of gestures is determined to be six, then any one of the gestures provided in FIGS. 8A-8K can count towards the threshold. When six gestures are counted, then the threshold level is met, and the person's hands are determined to be clean. In each of the aforementioned implementations, the system 100 can provide that the hands are not clean when the number of gestures does not meet the threshold.

Some implementations of the present disclosure can help educate the population to limit spread of virus in their families, avoid post-containment relapse and improve overall hygiene of the population in the future. Some implementations of the present disclosure can be used by private nurses and health professionals as a reminder for WHO or CDC best practices. Communication provided by some implementations of the present disclosure allow healthcare workers like nurses to devote attention to other medical actions. Educational videos provide help for fragile people or people unable to speak the local language, thus alleviating additional stress from caregivers. Proper handwashing can reduce or prevent the spread of communicable diseases, hence some implementations of the present disclosure are geared towards stemming the spread of disease.

Some implementations of the present disclosure can be used in a toy for kids to encourage and instill proper handwashing habits.

One or more elements or aspects or steps, or any portion(s) thereof, from one or more of any of claims 1-21 below can be combined with one or more elements or aspects or steps, or any portion(s) thereof, from one or more of any of the other claims 1-21 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving data associated with an individual, the individual having hands;
determining, from the received data, that the individual is washing the hands; and
generating a response to be communicated to the individual based at least in part on the determination that the individual is washing the hands.

2. The method of claim 1, wherein the received data includes motion data, sound data, temperature data, image data, location data, calendar information, or any combination thereof, and wherein sound data and motion data are combined to determine a duration of the washing of the hands, a starting time of the washing of the hands, an ending time of the washing of the hands, or any combination thereof.

3. (canceled)

4. The method of claim 1, wherein the generated response includes a notification indicating a remaining duration for the washing of the hands.

5. The method of claim 1, further comprising:
determining, from the received data, one or more gestures associated with washing the hands;
determining that a number of the one or more gestures determined exceeds a threshold; and
determining that the hands are clean based at least in part on the number of the one or more gestures exceeding the threshold.

6. The method of claim 5, wherein the response includes an indication that the hands are clean.

7. The method of claim 6, further comprising:
determining that a duration of the washing of the hands exceeds a duration threshold; and
wherein the determining that the hands are clean is further based at least in part on the duration of the washing of the hands.

8. The method of claim 1, further comprising:
determining, from the received data, one or more gestures associated with washing the hands;
determining that a number of the one or more gestures determined is below a threshold; and
determining that the hands are not clean based at least in part on the number of the one or more gestures exceeding the threshold.

9. The method of claim 8, wherein the response includes an indication that the hands are not clean.

10. The method of claim 9, further comprising:
determining that a duration of the washing of the hands is below a duration threshold; and
wherein the determining that the hands are not clean is further based at least in part on the duration of the washing of the hands.

11. The method of claim 1, wherein the data is received from a smartwatch, a bracelet, a smartphone, or any combination thereof.

12. A method comprising:
receiving data associated with an individual, the individual having hands;
determining, from the received data, that the individual should wash the hands; and
generating a response to be communicated to the individual based at least in part on the determination that the individual should wash the hands.

13. The method of claim 12, wherein the generated response includes a notification indicating a remaining duration for the washing of the hands.

14. The method of claim 13, wherein the determining that the individual should wash the hands is based at least in part on GPS data, a calendar of the individual, a time duration from a last time that the hands were washed, or any combination thereof.

15. A system for monitoring handwashing comprising:
a sensor configured to generate data associated with an individual, the individual having hands;
a memory storing machine-readable instructions; and
a control system including one or more processors configured to execute the machine-readable instructions to:
determine, from the received data, that the individual is washing the hands; and
generate a response to be communicated to the individual based at least in part on the determination that the individual is washing the hands.

16. The system of claim 15, wherein the sensor is coupled to a smartwatch, a smartphone, a bracelet, or any combination thereof.

17. The system of claim 16, wherein the sensor comprises one or more cameras, one or more infrared cameras, one or more motion sensors, one or more temperature sensors, one or more accelerometers, one or more magnetometers, one or more microphones, or any combination thereof.

18. The system of claim 17, wherein the sensor is coupled to a faucet, a mirror, a fixture, or any combination thereof.

19. The system of claim 18, wherein gestures indicating relative positioning of the hands are determined based at least in part on the generated data.

20. The system of claim 19, wherein the control system is further configured to execute the machine-readable instructions to determine whether a number of the determined gestures exceeds a threshold, and based on the number of the determined gestures exceeding the threshold, determining that the hands are clean.

21. The system of claim 19, wherein the control system is further configured to execute the machine-readable instructions to determine whether a number of the determined gestures exceeds a threshold, and based on the number of the determined gestures not exceeding the threshold, determining that the hands are unclean.
